(19) United States
(12) Patent Application Publication
(10) Pub. No.: US 2011/0288509 A1
(43) Pub. Date: Nov. 24, 2011

(54) MULTI-BANDAGE

(75) Inventors: Bernard Bar-Natan, Givatayim (IL); Michal Stadler Bekerman, Gedera (IL); Amnon Hamdani, Ashdod (IL); Graham Hill, London Greater London (GB)

(73) Assignee: FIRST CARE PRODUCTS LTD., Lod (IL)

(21) Appl. No.: 13/001,999

(22) PCT Filed: Jul. 2, 2009
(86) PCT No.: PCT/IL2009/000664

§ 371 Date: Aug. 2, 2011

(30) Foreign Application Priority Data
Jul. 2, 2008 (IL) 192579

(51) Int. Cl. A61M 35/00; A61F 13/64

(52) U.S. Cl. 604/308; 604/392

(57) ABSTRACT

There is provided herein a disparate use bandage comprising a pad portion and an elongate wrapping portion attached at an end thereof to an edge of said pad portion, said pad portion comprising an outer layer, at least one absorbent layer, an inner layer formed of a generally non-permeable material which is attached to the remainder of said pad portion in a readily removable manner.

### FIELD OF THE INVENTION

The invention generally relates to bandages and methods of using same.

### BACKGROUND

Various types of bandages are known in the literature and markets. The following publications are believed to represent the current state of the art: U.S. Pat. Nos. 34,112; 721,162; 2,113,534; 2,480,430; 2,646,034; 3,005,454; 3,050,064; 3,536,068; 4,048,991; 4,149,540; 4,243,028; 4,345,591; 4,802,667; 5,234,459 and 5,628,723.

Among the purposes of bandages are protecting exposed wounds and absorbing blood or other secretions. Bandages may be packaged to be carried and used for example, by military personnel, police, ambulances, hikers and campers, and may also be part of first-aid kits, for use in clinics, hospitals, factories, offices and the home.

Bandages, particularly with large bandages, are often not available in large quantities due to volume they occupy, particularly when they are carried by medics, doctors or other first-aid providers.

One of the problems thus associated with bandages, particularly with large bandages, is that different conditions or injuries require different treatment protocols. For example, injuries such as burns and abdomen injuries which involve the exposure of internal organs, the bandage is required to retain the moisture within the wound area, while in other injuries, such as amputations, the same type of bandage is required to absorb blood and secretions. Another example of a problem associated with large bandages, is that the same type of bandage should be used to dress an abdomen, for example, which involves wrapping the bandage around the subject, and to dress a limb which is much narrower than the abdomen.

There is thus a need in the art for improved bandages, particularly large bandages.

### SUMMARY

This summary section of the patent application is intended to provide an overview of the subject matter disclosed herein, in a form lengthier than an “abstract”, and should not be construed as limiting the invention to any features described in this summary section.

Some embodiments of the invention are generally directed to an improved bandage which has multiple, disparate applications.

There is thus provided, in accordance with some embodiments of the invention, a disparate use bandage including a pad portion and an elongate wrapping portion attached at an end thereof to an edge of the pad portion, the pad portion includes an outer layer, at least on absorbent layer and an inner layer formed of a generally non-permeable material which is attached to the remainder of the pad portion in a readily removable manner.

Preferably, the inner layer is attached to the remainder of the pad portion in a plurality (two or more) of points (such as sewn, glued, melted, stapled, pinned and the like) points. According to some embodiments, the sewn points may extend alongside and inwardly of seams of the pad portion.

In a preferred embodiment, the at least one absorbent layer is formed of a non-woven material.

Preferably, the inner layer includes plastic. Additionally or alternatively, the inner layer includes silicon. The inner layer may be smaller than the pad portion, essentially the same size as the pad portion or larger than the pad portion. According to some embodiments, the inner layer is adapted to prevent the adhesion of the bandage (for example the pad portion) to the body tissue (such as, the burn, the intestine or any other body tissue).

In a preferred embodiment, the pad portion may also include a blood dispersing barrier layer between the outer layer and the at least one absorbent layer.

Preferably, the pad portion also includes a sterile non-adherent layer between at least one absorbent layer and the inner layer.

According to some embodiments, the disparate use bandage may further include one or more wrapping element adapted to facilitate changing a wrapping direction of said elongate wrapping portion.

According to some embodiments, the disparate use bandage may further include an opening adapted to facilitate application of a medical substance to a wounded area. The substance may include saline, water, a medicament or any combination thereof. The medicament may include analgesics, anti-inflammatory drugs, antibiotics, anti-septic drugs or any combination thereof.

According to some embodiments, the disparate use bandage may further include a retaining device adapted to maintain said elongate wrapping portion in a rolled or folded configuration, allowing a user to gradually unroll or unfold the web, without the remaining elongate wrapping portion unrolling or unfolding itself entirely.

### BRIEF DESCRIPTION OF FIGURES

Examples illustrative of embodiments of the invention are described below with reference to figures attached hereto. In the figures, identical, structures, elements or parts that appear in more than one figure are generally labeled with a same numeral in all the figures in which they appear. Dimensions of components and features shown in the figures are generally chosen for convenience and clarity of presentation and are not necessarily shown to scale. The figures (FIGs.) are listed below.

FIGS. 1A and 1B are simplified respective pictorial and sectional illustrations of a disparate use bandage constructed and operative in accordance with some embodiments of the present invention, in a first operative orientation;

FIGS. 2A and 2B are simplified respective pictorial and sectional illustrations of the bandage of FIGS. 1A and 1B, in a second operative orientation;

FIG. 3 is a simplified illustration of a use the bandage of FIGS. 1A and 1B;

FIG. 4 is a simplified illustration of a use the bandage of FIGS. 2A and 2B;

FIGS. 5A-5D are simplified illustrations of a bandage and the use thereof, in accordance with some embodiments of the present invention;

FIGS. 6A and 6B illustrate bandages, constructed and operative in accordance with embodiments of the present invention;

FIG. 7 illustrates a bandage, constructed and operative in accordance with embodiments of the present invention; and

FIGS. 8A and 8B are cross-sectional illustrations of retained rolled and folded elongate wrapping portions (web portions), in accordance with embodiments of the present invention.

### DETAILED DESCRIPTION OF A PREFERRED EMBODIMENTS

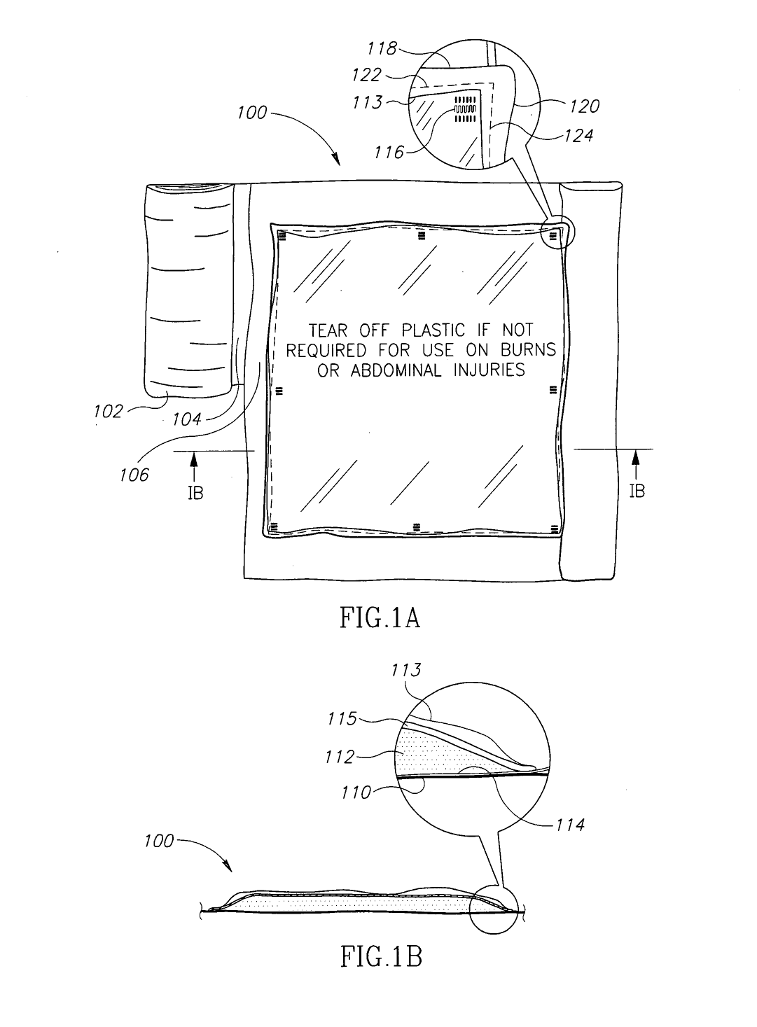

Reference is now made to FIGS. 1A and 1B, which are simplified respective pictorial and sectional (along line IB) illustrations of a disparate use bandage constructed and operative in accordance with a preferred embodiment of the present invention in a first operative orientation. As seen in FIGS. 1A and 1B, the bandage comprises a generally square (can also be rectangular or any other shape) pad portion 100 and an elongate wrapping portion 102 (may also be referred to as a web portion) attached at one and 104 thereof to an edge 106 of pad portion 100.

Pad portion 100 preferably has dimensions of 30 cm by 30 cm and preferably comprises an outer layer 110, preferably a layer similar to elongate wrapping portion, one or more (or a plurality of) absorbent layers 112, typically non-woven of cotton, and an inner layer 113 of a generally non-permeable non-adherent material, such as plastic or silicon or any other suitable material. Pad portion 100 may also include a blood dispersing barrier layer 114, preferably made of plastic, between outer layer 110 and absorbent layers 112. Pad portion 100 may also include a sterile non-adherent layer 115, preferably formed of a layer of non-woven cloth adherent thereto, between absorbent layers 112 and inner layer 113.

It is appreciated that elongate wrapping portion 102 may be integrally formed with outer layer 110.

It is a particular feature of the present invention that the inner layer 113 is attached to the remainder of pad portion 100 in a readily removable manner, such as by means of a limited number of sewn points 116 adjacent edges 118 and 120 thereof. Inner layer 113 may be attached to pad portion 100 by other readily removable manners, such as by gluing, welding (for example heat welding), stapling or any other way.

Preferably, the attachment points, such as sewn points 116, extend, as shown, alongside and inwardly of seams 122 and 124 along edges 118 and 120.

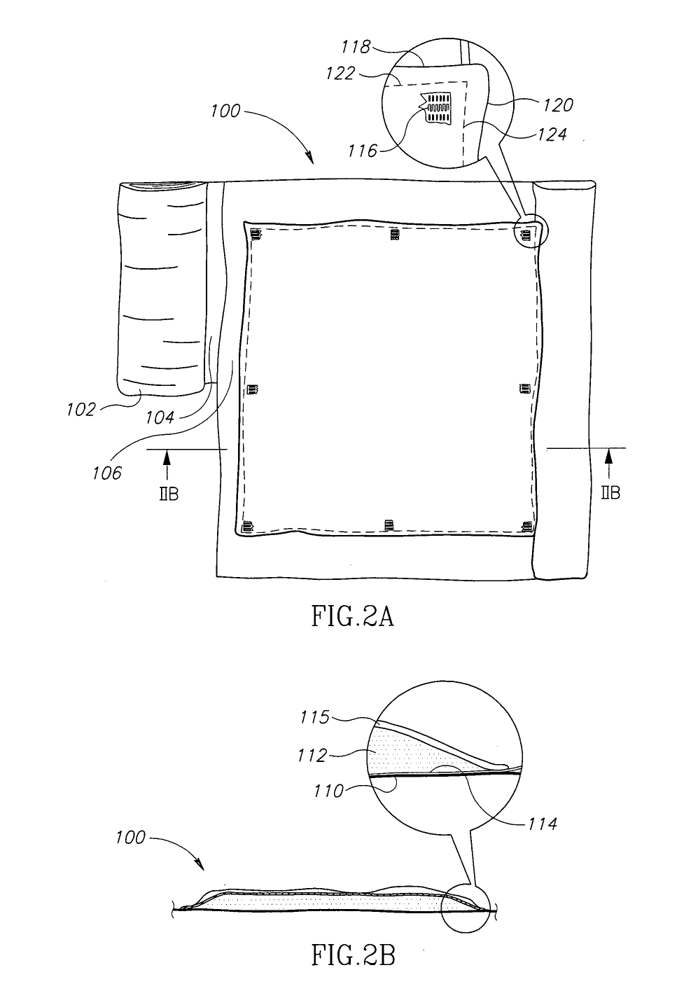

Reference is now made to FIGS. 2A and 2B, which are simplified respective pictorial and sectional (along line IIB) illustrations of the bandage of FIGS. 1A—and 1B in a second operative orientation. As can be readily seen in FIGS. 2A and 2B, here, the non-permeable inner layer 113 is not present, having earlier been removed as by pulling and being separated from the remainder of the pad portion 100 at sewn points 116.

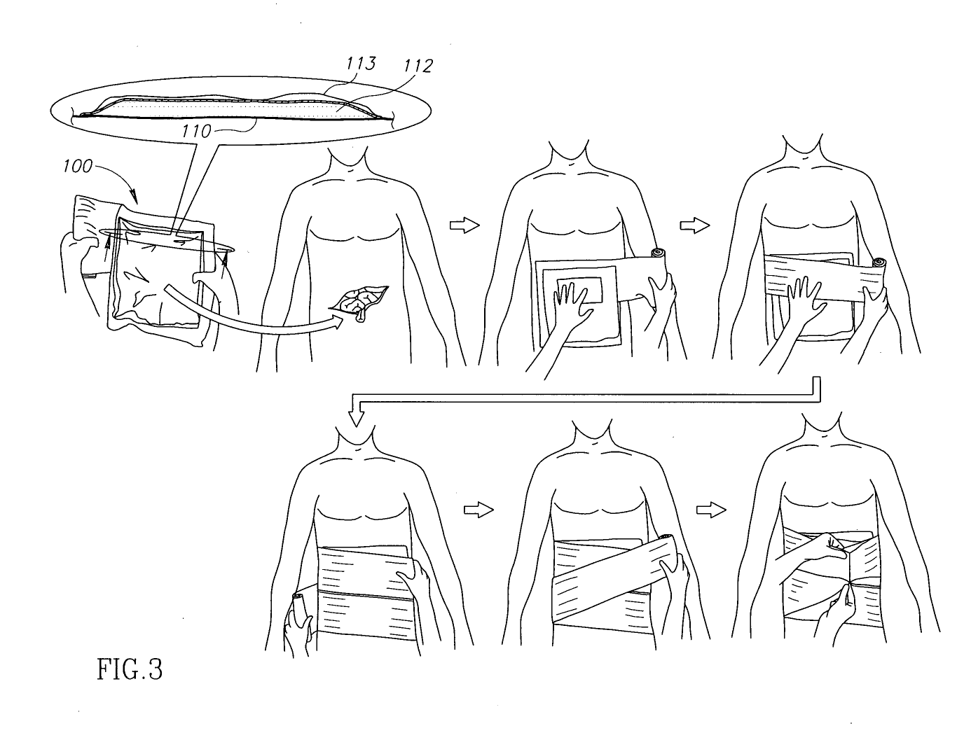

FIG. 3 shows a preferred use of the bandage of the present invention in the operative state shown in FIGS. 1A and B, for emergency treatment of abdominal injuries, wherein the non-permeable layer is essential for proper wound treatment. It is appreciated that the bandage including the non-permeable inner layer 113 preferably is provided for treatment of a wound where the retention of moisture therein is indicated, such as in the case of exposure of internal organs (such as the intestines) and burns. Inner layer 113 is also characterized in non-adhesiveness. Non-permeable inner layer 113 will preferably not be removed from the bandage, prior to use thereof, in situations where a bandage with this property is indicated to promote proper wound treatment, such as in the case of burns or exposed internal organs.

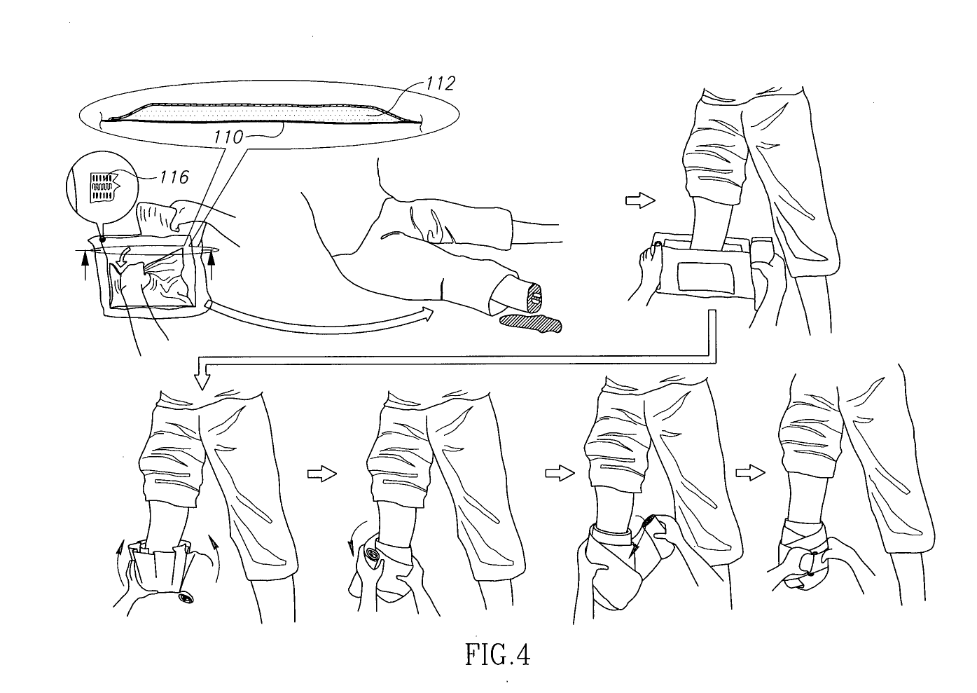

FIG. 4 shows a preferred use of the bandage of the present invention in the operative state shown in FIGS. 2A and 2B for emergency treatment of severe injuries including amputations and large surface area traumatic wounds, such as due to an explosion. Here it is important that the non-permeable layer not be present.

It is appreciated that the bandage following the removal of the non-permeable inner layer 113 preferably is provided for treatment of a wound where moisture absorption by the bandage is indicated. Non permeable inner layer 113 will preferably be removed from the bandage, prior to use thereof in situations where a bandage with this property is indicated to promote proper wound treatment.

It is appreciated that the bandage, in accordance with some embodiments of the present invention, may be used in either orientation in situations where a large surface area covering is required to be applied to the wound and surrounding area.

As seen in FIGS. 3 and 4, the bandage of the present invention provides a disparate use bandage that can be applied in the appropriate orientation by the attending medical professional.

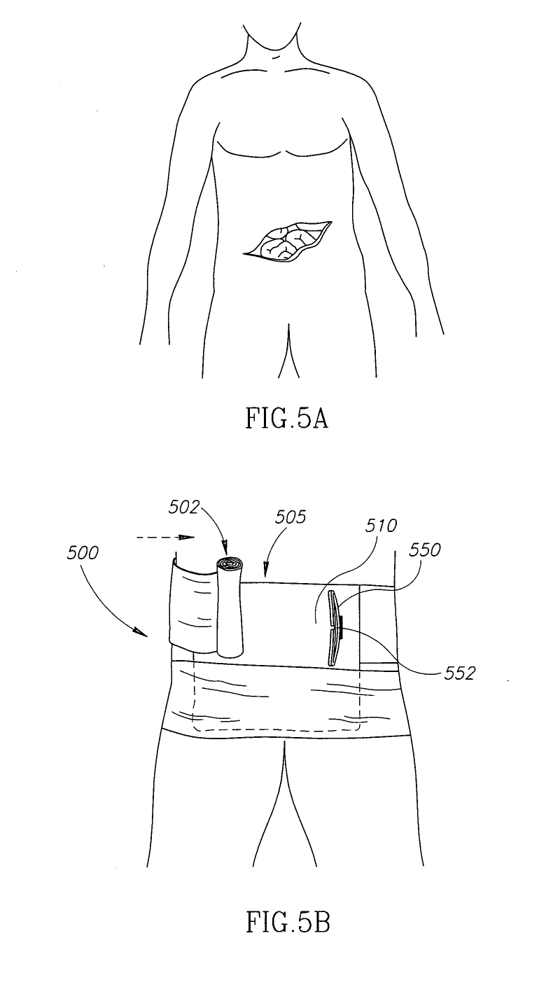

Reference is now made to FIGS. 5A-5D which show simplified illustrations of a bandage and the use thereof, in accordance with some embodiments of the present invention. FIG. 5A shows a simplified illustration of a part of a subject having an abdominal injury with exposed intestines which requires dressing. FIG. 5B shows a simplified illustration of bandage 500 covering the wound and wrapped around the subject in a first direction (indicated by the dashed arrow). Bandage 500 has a pad portion 505 including an inner layer (not shown) covering the wound, wherein the inner layer (which may be similar to inner layer 113 in FIG. 1B) is adapted to maintain the moisture within the wound area. Pad portion 505 also includes outer layer 510 which continues to (may also be attached to) elongate wrapping portion 502. Elongate wrapping portion 502 (which may also be referred to as a web portion) may be made of an elastic material to facilitate wrapping around the subject. Bandage 500 further includes wrapping element 550 shown here in as located on a top corner of pad portion 505. Wrapping element 550 may also be located at a bottom corner of pad portion 505 or anyplace else. In addition, bandages such as bandage 500 may include two or more wrapping element such as wrapping element 550, for example on two adjacent corners of pad portion 505 or o two opposite corners of pad portion 505. Wrapping element 550 includes gap 552 adapted to allow elongate wrapping portion 502 enter a space 534 wrapping element 550 when wrapping elongate wrapping portion 502, in a first direction, around the subject or around the wounded organ (as shown in FIG. 5C). Elongate wrapping portion 502 is then wrapped around the subject or around the wounded organ in a second direction (as shown in FIG. 5D). Wrapping in first direction and then using wrapping element 550 for wrapping in a second direction “saves” a “wrapping round” which thus saves web portion material and also saves and time and inconvenience to the wounded subject, particularly in a case of large organ injury, such as the abdomen.

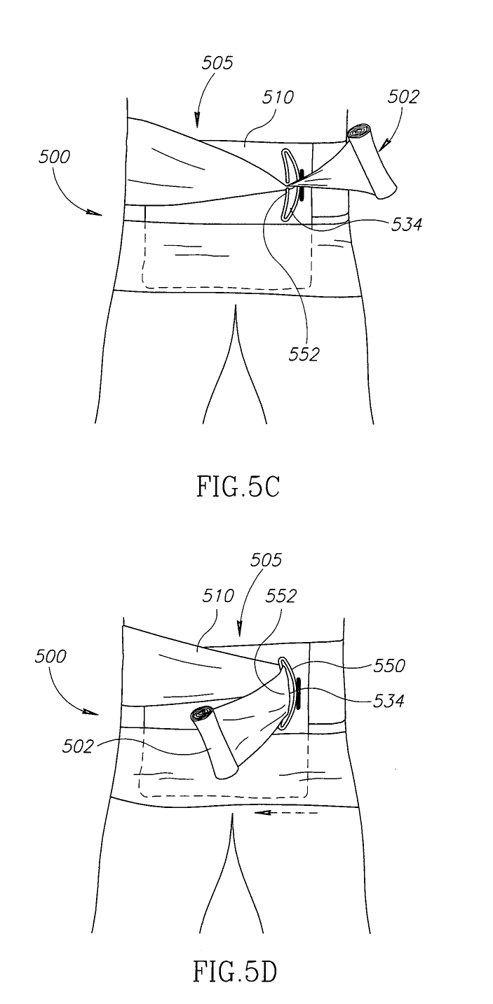

In accordance with some embodiments of the present invention, the wrapping element may have any shape or form that would be appropriate for facilitating the wrapping of the bandage. Reference is now made to FIGS. 6A and 6B which illustrate bandage 600 and bandage 1600, respectively, constructed and operative in accordance with other embodiments of the present invention. Bandage 600 of FIG. 6A includes a pad portion 605, an elongate wrapping portion (web portion) 604 and a wrapping element 650. Wrapping element 650 is preferably made of a flexible, resilient material, such as a structural plastic, and includes a bow-shaped portion 620 fixedly attached to a base 622 and generally perpendicular thereto. Two arcuate upper wings 624 are joined to bow-shaped portion 620 at rounded junctions 626. Bow-shaped portion 620 and upper wings 624 form a wrapping element. Wrapping element 650 is mounted on pad portion 605 such that upper wings 624 and bow-shaped portion 620 curve away from pad portion 605. Near each rounded junction 626 there may optionally be an arcuate stiffening rib 628. Upper wings 624 are preferably chamfered at tips 629 which are separated from each other by a gap 652. Upper wings 624 and bow-shaped portion 620 define therebetween a space 634. Similar to what is shown in FIG. 5 A-D, elongate wrapping portion (web portion) 604 is pressed against upper wings 624 such that elongate wrapping portion (web portion) 604 is pushed through gap 652 into space 634. The resiliency of upper wings 624 facilitates pushing elongate wrapping portion (web portion) 604 through gap 652. Chamfered tips 629 also facilitate pushing elongate wrapping portion (web portion) 604 through gap 652. More optional shapes of wrapping elements may be found in international patent application, publication number WO 97/29689 (Grau Bernard), which is incorporated herein by reference in its entirety.

Bandage 1600 of FIG. 6B includes a pad portion 1605, an elongate wrapping portion (web portion) 1604 and two wrapping elements 1650 and 1660, which are, in this case, similar but may also be different from one another. Wrapping elements 1650 and 1660 are disposed at two sides of pad portion 1605 and adapted to facilitate wrapping of elongate wrapping portion (web portion) 1604. Wrapping element 1650 includes: a bow-shaped portion 1620, a base 1622, two arcuate upper wings 1624 joined to bow-shaped portion 1620 at rounded junctions 1626, arcuate stiffening rib 1628, gap 1652, space 1634 and chamfered tips 1629 are similar in their structure and functionality to bow-shaped portion 620, a base 622, two arcuate upper wings 624 joined to bow-shaped portion 620 at rounded junctions 626, arcuate stiffening rib 628, gap 652, space 634 and chamfered tips 629 of wrapping element 650 of FIG. 6A.

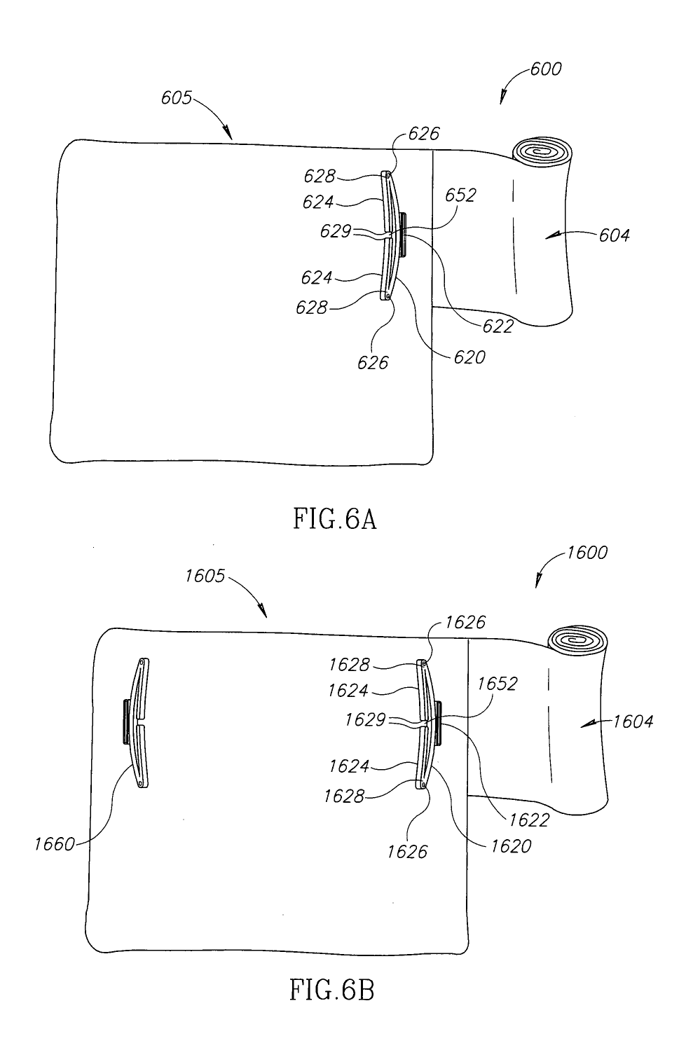

Reference is now made to FIG. 7 which illustrates a bandage 700, constructed and operative in accordance with other embodiments of the present invention. Bandage 700 includes a pad portion 705 including absorbent layer(s) and optionally an inner layer and blood dispersing barrier layer (not shown here but may be similar to the layers described in FIG. 1B or FIG. 2B). Pad portion 705 also includes outer layer 710 which continues to (may also be attached to) elongate wrapping portion 702. Pad portion 705 further includes an arcuate opening 760 adapted to facilitate application of liquid (such as saline or water) and or medications (such as analgesics, anti-inflammatory drugs, antibiotics, anti-septic drugs or any other medication) directly to the wounded tissue. For example, in the case of abdominal injury with exposed intestine, it may be recommended, if the conditions and environment allow it, to wet the exposed intestine with saline of even water and optionally to topically apply drugs to the wound.

According to additional embodiments of the invention, the elongate wrapping portions (web portions) of the bandaged disclosed herein may be maintained in a rolled or folded configuration, by one or more “retaining device”, while allowing the user (person applying the bandage, caregiver and/or wounded subject) to easily and gradually unroll or unfold the web, sequentially, such as step-by-step, as needed, without the remaining web unrolling or unfolding itself and dropping to the ground.

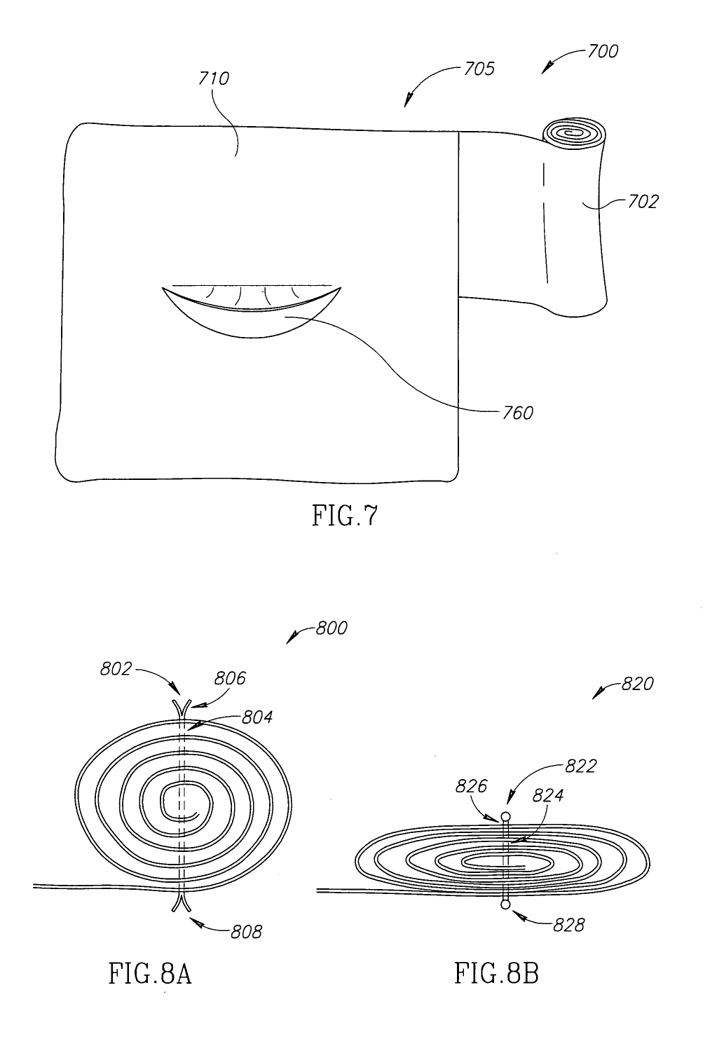

Reference is now made to FIGS. 8A and 8B which show cross-sectional illustrations of retained rolled 800 and folded 820 elongate wrapping portions (web portions) of a bandage, in accordance with embodiments of the present invention. Rolled elongate wrapping portion 800 is retained by a retaining device 802 and folded elongate wrapping portion 820 is retained by a retaining device 822.

Retaining devices 802 includes an elongated element 804, a top end element 806 (having a V shape) and a bottom end element 808 (also having a V shape) opposing to top end element 806, wherein retaining device 802 is adapted to extend through two or more panels of rolled elongate wrapping portion 800, and releasably retain elongate wrapping portion 800 in a rolled configuration. The device may be further adapted to allow a user of the bandage to gradually unroll without removing the device by pulling one or more panels of elongate wrapping portion 800 over top end element 806, while other panels remain retained.

Retaining devices 822 includes an elongated element 824, a top end element 826 (having a ball shape) and a bottom end element 828 (also having a V shape) opposing to top end element 806, wherein retaining device 822 is adapted to extend through two or more panels of folded elongate wrapping portion 820, and releasably retain elongate wrapping portion 820 in a folded configuration. The device may be further adapted to allow a user of the bandage to gradually unfold without removing the device by pulling one or more panels of elongate wrapping portion 820 over top element 826, while other panels remain retained.

The retaining device may be made of cotton, synthetic fibers, natural fibers, plastic, nylon, silicon or any other appropriate material. More details about potential retaining devices may be found in Israeli Patent Application No. 197854, which is incorporated by reference herein in its entirety.

In the description and claims of the application, each of the words “comprise” “include” and “have”, and forms thereof, are not necessarily limited to members in a list with which the words may be associated.

It will be appreciated by persons skilled in the art that the present invention is not limited by what has been particularly shown and described hereinabove. Rather the scope of the invention includes both combinations and sub combinations of various features described hereinabove as well as modifications and variations thereof which would occur to a person skilled in the art upon reading the foregoing description and which are not in the prior art.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art. It is intended that the scope of the invention be limited only by the claims and that the claims be interpreted to include all such variations and combinations.

What is claimed is:

1. A disparate use bandage comprising:
a pad portion; and
an elongate wrapping portion attached at an end thereof to an edge of said pad portion, said pad portion comprising an outer layer, at least one absorbent layer, an inner layer formed of a generally non-permeable material which is attached to the remainder of said pad portion in a readily removable manner.

2. A disparate use bandage according to claim 1 further comprising one or more wrapping elements adapted to facilitate changing a wrapping direction of said elongate wrapping portion.

3. A disparate use bandage according to claim 1 further comprising one wrapping element located at a corner of the pad portion, the wrapping element is adapted to facilitate changing a wrapping direction of said elongate wrapping portion.

4. A disparate use bandage according to claim 1 further comprising two wrapping elements located at two opposing corners of the pad portion, the wrapping elements are adapted to facilitate changing a wrapping directions of said elongate wrapping portion.

5. A disparate use bandage according to claim 1 further comprising an opening adapted to facilitate application of a medical substance to a wounded area.

6. A disparate use bandage according to claim 5 and wherein the substance comprising saline, water, a medicament or any combination thereof.

7. A disparate use bandage according to claim 6 and wherein the medicament comprises analgesics, anti-inflammatory drugs, antibiotics, anti-septic drugs or any combination thereof.

8. A disparate use bandage according to claim 1 and wherein said inner layer is attached to the remainder of said pad portion by a plurality of sewn points.

9. A disparate use bandage according to claim 8 and wherein said sewn points extend alongside and inwardly of seams of said pad portion.

10. A disparate use bandage according to claim 1 and wherein said inner layer is attached to the remainder of said pad portion by a plurality of glued points.

11. A disparate use bandage according to claim 1 and wherein said at least one absorbent layer is formed of a non-woven material.

12. A disparate use bandage according to claim 1 and wherein said inner layer comprises plastic.

13. A disparate use bandage according to claim 1 and wherein said inner layer comprises silicon.

14. A disparate use bandage according to claim 1 and wherein said pad portion also comprises a blood dispersing barrier layer between said outer layer and said at least one absorbent layer.

15. A disparate use bandage according to claim 1 and wherein said pad portion also comprises a non-adherent layer between said at least one absorbent layer and said inner layer.

16. A disparate use bandage according to claim 1 further comprising a retaining device adapted to maintain said elongate wrapping portion in a rolled or folded configuration, allowing a user to gradually unroll or unfold the web, without the remaining elongate wrapping portion unrolling or unfolding itself entirely.
